(19) United States
(12) Patent Application Publication
(10) Pub. No.: US 2026/0315696 A1
(43) Pub. Date: Oct. 15, 2026

(54) A sub-assembly for a medicament delivery device

(71) Applicant: SHL Medical AG, Zug (CH)

(72) Inventors: Anders Boström, Ingarö (SE); Oscar Alexandersson, Nacka Strand (SE)

(21) Appl. No.: 19/485,310

(22) PCT Filed: May 24, 2024
(86) PCT No.: PCT/EP2024/064323

§ 371 Date: Nov. 18, 2025

(30) Foreign Application Priority Data
Jun. 6, 2023 (EP) 23177460.5

(51) Int. Cl. A61M 5/20

(52) U.S. Cl. CPC A61M 5/2033

(57) ABSTRACT

The present disclosure provides a sub-assembly for a medicament delivery device configured to expel medicament from a medicament container. The sub-assembly includes a housing, a plunger rod, and a pre-tensioned rotatable driver arrangement for the plunger rod being operably arranged to, upon activation, rotate and thereby move the plunger rod proximally to act on the medicament container for expelling a medicament. The rotatable driver arrangement includes a contacting surface and a braking button having an attachment surface, the braking button being operatively arranged to, upon activation, move to a second state in which the attachment surface is abutting the contacting surface of the rotatable driver arrangement to thereby brake the rotational movement of the rotatable driver arrangement and slow down the proximal movement of the plunger rod.

### TECHNICAL FIELD

The present disclosure generally relates to medical devices for medicament administration.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology.

The medicament is typically comprised in a medicament container within the medicament delivery device, the medicament container being configured to expel the medicament via some type of delivery member, such as a needle or a nozzle. A medicament delivery action may be activated by releasing a pre-tensioned plunger rod configured to act on the medicament container, whereafter medicament is continuously expelled from the medicament container until the end of the medicament delivery action. However, a user may want to control the medicament delivery action subsequent to that the medicament delivery action has been activated.

In considering these problems, the applicant has appreciated that various developments could be made to help improve the medicament delivery devices on the market today, which are set out in more detail below.

### SUMMARY

An object of the present disclosure is to provide a sub-assembly for a medicament delivery device, and a medicament delivery device, which solves, or at least mitigates problems of the prior art.

There is hence provided a sub-assembly for a medicament delivery device configured to expel medicament from a medicament container. The sub-assembly comprises: a housing having a proximal end and a distal end; a plunger rod arranged inside the housing; a pre-tensioned rotatable driver arrangement for the plunger rod, the rotatable driver arrangement being operably arranged to, upon activation, rotate and thereby move the plunger rod proximally to act on the medicament container for expelling a medicament, the rotatable driver arrangement having a contacting surface; a braking button having an attachment surface, the braking button being operatively arranged to, upon activation, move from a first state in which the attachment surface is distant from the contacting surface of the rotatable driver arrangement, to a second state in which the attachment surface is abutting the contacting surface of the rotatable driver arrangement to thereby brake the rotational movement of the rotatable driver arrangement and slow down the proximal movement of the plunger rod.

The speed of the proximal movement of the plunger rod can thus be reduced in an improved manner. Thereby, the expel of medicament from the medicament container can be adapted accordingly. For example, the speed of expelling medicament from the medicament container can be reduced by activating the braking button and reducing the rotation of the rotatable driver arrangement. By providing a braking button being arrangeable in a first state in which the attachment surface is distant from the contacting surface of the rotatable driver arrangement, and a second state in which the attachment surface is abutting the contacting surface of the rotatable driver arrangement, an efficient means for applying a braking torque to the rotatable driver arrangement is provided. Typically, the braking torque is achieved by friction between the contacting surface and the attachment surface. In more detail, as the friction between the braking button and the rotatable driver arrangement is increased from the first state of the braking button to the second state of the braking button, the rotation of the rotatable driver arrangement is slowed down owing to the increased friction. The physical action of moving the braking button from the first state to the second state can be achieved by a user of the sub-assembly. Thus, the braking button is preferably configured to be manually operated by the user of the sub-assembly.

By means of the braking button, the user of the sub-assembly can adapt the expelling speed of medicament from the medicament container.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment, the braking button is configured to, in the second state, brake the rotatable driver arrangement by friction. Thus, as the attachment surface of the braking button is brought into contact with the contacting surface of the rotatable driver arrangement, friction between the attachments surface and the contacting surface acts to slow down the rotation of the rotatable driver arrangement.

According to one embodiment, the braking button is configured to vary the friction between the attachments surface and the contacting surface as a function of an applied force on, or to, the braking button. For example, by applying a larger force to the braking button, typically by the user of the sub-assembly, the friction between the attachments surface and the contacting surface is increased and the rotation of the rotatable driver arrangement is further slowed down. Correspondingly, by applying a smaller force to the braking button, typically by the user of the sub-assembly, the friction between the attachments surface and the contacting surface is decreased and the rotation of the rotatable driver arrangement is slowed down to a lesser extent as compared to when the larger force is applied. For example, the varied friction between the attachments surface and the contacting surface can be achieved by varying the pressure of the attachments surface harder against the contacting surface, or by varying the contacting surface area between the attachments surface and the contacting surface.

According to one embodiment, the rotatable driver arrangement is configured to act on the plunger rod to move the plunger rod proximally. This may e.g. be achieved by that the rotatable driver arrangement is rotationally locked to the plunger rod. Such rotational lock may e.g. comprise a male-female structure, e.g. in the form of a protrusion of the rotatable driver arrangement connecting with an indentation of the plunger rod. Moreover, the sub-assembly, or medicament delivery device, may comprise a feeding structure arranged radially outside of the plunger rod. Such feeding structure is preferably arranged proximal of the rotatable driver arrangement. The feeding structure may be configured to move, or feed, the plunger rod proximally upon rotation of the plunger rod. For example, the feeding structure and the plunger rod are configured to cooperate in a screw-nut connection. For example, the plunger rod may comprise an external thread, and the feeding structure may comprise an internal thread configured to mate with the external thread of the plunger rod. Thus, a rotational movement of the plunger rod will result in that the plunger rod is moved axially relative to the feeding structure. The feeding structure may e.g. be rotationally fixed to the housing, and/or may be attached to another internal component of the sub-assembly which can be held rotationally fixed in relation to the plunger rod.

According to one embodiment, the braking button is, in the first state, arranged to prevent affecting the rotational movement of the rotatable driver arrangement. That is, in the first state, there may be no contact between the braking button and the rotatable driver arrangement. However, according to an alternative embodiment, the braking button is in contact with the rotatable driver arrangement in the first state, but not by contact via the attachment surface and the contacting surface.

According to one embodiment, the braking button extends radially and/or axially from outside of the housing to inside of the housing. Thus, a user may access the braking button from outside of the housing, typically to apply a force to the braking button, whereby the braking button may affect the rotational driver arrangement inside of the housing.

According to one embodiment, the rotatable driver arrangement comprises a tubular main driver holding the plunger rod, the tubular main driver having an inner facing interacting surface and the plunger rod having an outer facing interacting surface, wherein the inner and outer facing interacting surfaces are arranged to directly interact to transform a rotational movement of the tubular main driver to a rotational movement of the plunger rod. That is, the tubular main driver is hollow, and comprises a cylindrically shaped inner bore, wherein surface(s) of the inner bore facing towards a centre axis of the tubular main driver comprises, or forms, the inner facing interacting surface. Correspondingly, the plunger rod being configured to be move within the cylindrically shaped inner bore of the tubular main driver, comprises surface(s) facing away from a centre axis of the plunger rod, wherein such surface(s) comprises, or forms, the outer facing interacting surface.

According to one embodiment, the inner facing interacting surface comprises an inner thread of the tubular man driver, and the outer facing interacting surface comprises an outer thread, wherein the inner thread is configured to threadingly mate with the outer thread to transform a rotational movement of the tubular main driver to a rotational movement of the plunger rod. As previously described, the plunger rod is configured to move proximally upon rotation. In order to rotationally lock the tubular main driver to the plunger rod, the previously mentioned protrusion of the rotatable driver arrangement (connecting with the indentation of the plunger rod) may be arranged on the tubular main driver, and the indention of the plunger rod may be an axially and radially extending indentation, e.g. extending from the proximal end to the distal end of the plunger rod.

According to one embodiment, the contacting surface of the rotatable driver arrangement is arranged on the tubular main driver, wherein in the second state of the activation button, the attachment surface is abutting the contacting surface of the tubular main driver to thereby directly brake the rotational movement of the tubular main driver. That is, upon activation of the braking button, i.e. movement of the braking button from its first state to its second state, the attachment surface is moved internally inside the housing to abut the contacting surface of the tubular main driver. Hereby, friction between the attachments surface and the contacting surface results in braking of the rotational movement of the tubular main driver.

According to one embodiment, the contacting surface of the tubular main driver is comprised in an outer facing surface of the tubular main driver. Hereby, the braking button may interact with the tubular main driver in an efficient and improved manner. As previously described, the tubular main driver is hollow and comprises a cylindrically shaped inner bore, and thus comprises a main body surrounding the inner bore, wherein surface(s) of the main body facing away from the centre axis of the tubular main driver comprises, or forms, the outer facing surface. For example, in the first state of the braking button, the contacting surface faces, but do not contact, the attachment surface.

According to one embodiment, the contacting surface comprises a proximally facing surface portion, wherein the attachment surface of the braking button comprises a corresponding distally facing surface portion, such that in the second state of the activation button, the proximally facing surface portion of the contacting surface is abutting the distally facing surface portion of the attachment surface of the braking button. By the interaction of the proximally facing surface portion and the distally facing surface portion, friction causing the tubular main driver to brake may be applied in an advantageous manner. For example, radial and/or axial forces which may cause misalignment of components inside the housing, e.g. the tubular main driver, may be reduced. Stated differently, the outer facing surface of the tubular main driver may comprise the proximally facing surface portion, i.e. a portion of the outer facing surface making up the contacting surface has a portion which faces in the proximal direction. It should be understood that the proximally facing surface portion need not to have a normal component extending axially in the proximal direction, but it may be angled. That is, the normal component of the proximally facing surface portion may be angled relative to the centre axis. Correspondingly, the distally facing surface portion of the attachment surface need not to have a normal component extending axially in the distal direction, but it may be angled. That is, the normal component of the distally facing surface portion may be angled relative to the centre axis.

According to one embodiment, the contacting surface comprises an outer radial facing surface portion, wherein the attachment surface of the braking button comprises an inner radial facing surface portion, such that in the second state of the activation button, the outer radial facing surface portion of the contacting surface is abutting the inner radial facing surface portion of the attachment surface of the braking button. By the interaction of the outer radial facing surface portion and the inner radial facing surface portion, friction causing the tubular main driver to brake may be applied in an advantageous manner.

According to one embodiment, the contacting surface comprises the proximally facing surface portion and the outer radial facing surface portion, wherein the attachment surface of the braking button comprises the corresponding distally facing surface portion and the inner radial facing surface portion, such that in the second state of the activation button, the proximally facing surface portion of the contacting surface is abutting the distally facing surface portion of the attachment surface of the braking button, and the outer radial facing surface portion of the contacting surface is abutting the inner radial facing surface portion of the attachment surface of the braking button. By providing contact between both proximally and distally facing surface portions and inner and outer radially facing surface portions, friction causing the tubular main driver to brake may be applied in an advantageous manner. For example, radial and/or axial forces which may cause misalignment of components inside the housing, e.g. the tubular main driver, may be further reduced.

Stated differently, the outer facing surface of the tubular main driver may comprise the outer radial facing surface portion, i.e. a portion of the outer facing surface making up the contacting surface has a portion which faces in the radial direction, typically away from a centre axis of the tubular main driver. Typically, the outer radial facing surface portion has a normal component extending radially perpendicular to a centre axis of the tubular main driver. Correspondingly, the inner radial facing surface portion of the attachment surface typically has a normal component extending radially perpendicular and towards the centre axis of the tubular main driver.

According to one embodiment, the braking button comprises an elongated handle arranged radially outside of the housing, wherein the attachment surface is directly connected to a proximal end portion of the elongated handle. Hereby, the handle may act as a lever and improve the control of the braking button to the rotational driver arrangement. Thus, a user may access the braking button from radially outside of the housing by interacting with the handle, typically to apply a force to the handle acting as a lever relative to the attachment surface.

According to one embodiment, the braking button is a sliding button configured to move from the first state to the second state by axially sliding relative to the housing. Hereby, an efficient means for moving the braking button from the first state to the second state is provided. Moreover, by providing a sliding button configured to move axially, the attachment surface may be moved inside of the housing in an axial direction, and e.g. the previously mentioned proximally facing surface portion of the contacting surface may be brought to abut the distally facing surface portion of the attachment surface in an advantageous manner.

According to one embodiment, the rotatable driver arrangement further comprises a rotatable spinner arranged in a distal portion of the sub-assembly, the rotatable spinner being rotationally locked to the tubular main driver, wherein the contacting surface of the rotatable driver arrangement is arranged on the rotatable spinner, and wherein in the second state of the activation button, the attachment surface is abutting the contacting surface of the rotatable spinner to thereby indirectly brake the rotational movement of the tubular main driver. Hereby, the braking button may interact with the tubular main driver via the rotatable spinner in an efficient manner. That is, as the rotatable spinner is rotationally locked to the tubular main driver, by abutting the contacting surface of the rotatable spinner by the attachment surface of the braking button, the rotatable spinner is braked as well as the tubular main driver.

According to one embodiment, the braking button is a push-button configured to move from a protracted position in the first state to a retracted position in the second state in which the push-button is received further into the housing. Thus, a user may access the push-button from outside of the housing, typically to apply a force to the push-button in its protracted position, whereby the push-button may affect the rotational driver arrangement inside of the housing when being brought to its retracted position.

According to one embodiment, the housing comprises a sight window arranged in a distal portion of the housing, wherein the rotatable spinner is visible in a proximal direction through the sight window. Hereby, the rotatable spinner is made visible for a user, and the user may by its rotation receive feedback of the medicament delivery process.

It should be noted that the rotatable spinner, and optionally the sight window, may be comprised in the rotatable driver arrangement also for the embodiments in which the contacting surface of the rotatable driver arrangement is arranged on the tubular main driver.

According to one embodiment, the braking button is biased to its first state such that the braking button is operatively arranged to, upon de-activation, move from the second state to the first state in which the attachment surface is distant from the contacting surface of the rotatable driver arrangement to thereby decrease, or cease, the braking of the rotational movement of the rotatable driver arrangement and increase the proximal movement of the plunger rod. Hereby, the expelling speed of medicament from the medicament container may to at least some extent be adapted in response to the activation and de-activation of the braking button. By means of the biased braking button, the user of the sub-assembly can adapt the braking of the rotatable driver arrangement. The braking button may e.g. be biased by means of a spring, such as e.g. a leaf spring. Such spring or leaf spring is preferably tensioned against the housing, or a structure fixed to the housing.

According to one embodiment, the sub-assembly further comprises an activation sleeve fixedly attached to the housing, the activation sleeve comprising a holding arm arranged to abut a locking surface of the rotatable driver arrangement to lock the rotatable driver arrangement in its pre-tensioned state. Hereby, the rotatable driver arrangement can be arranged in its pre-tensioned state in an advantageous manner.

According to one embodiment, the sub-assembly further comprises a needle cover arranged to move axially relative the housing, the needle cover being operatively configured to distally push the activation sleeve into an activation position in which the holding arm is distant from the locking surface of the rotatable driver arrangement to thereby release the rotatable driver arrangement from its pre-tensioned state. Hereby, the rotatable driver arrangement can be released from its pre-tensioned state in an advantageous manner. For example, the activation sleeve is configured to move to the activation position by being moved distally. That is, as the blocking arm is moved distally upon the distal movement of the activation sleeve, the blocking arm is moved passed the locking surface, and the rotatable driver arrangement is released from its pre-tensioned state. The locking surface may according to one embodiment be arranged on the previously mentioned tubular main driver, e.g. at a proximal portion of the tubular main driver.

According to one embodiment, the rotatable driver arrangement is pre-tensioned by means of a torsion spring. For example, after activation of the rotatable driver arrangement, the torsion spring is configured to rotate the rotatable driver arrangement until the torsion spring becomes unbiased, or un-tensioned. However, as previously described, the braking button may act to brake the rotational movement of the rotatable driver arrangement.

According to one embodiment, the housing comprises a first housing part and a second housing part in the form of a rear cap. The rear cap may e.g. be removably attached to a distal end of the first housing part by a female-male connection, e.g. by a snap-fit connection.

According to a second aspect of the present disclosure, there is provided a medicament delivery device for expelling medicament from a medicament container, the medicament delivery device comprising the sub-assembly according to the first aspect.

Effects and features of the second aspect are largely analogous to those described above in connection with the first aspect. Embodiments mentioned in relation to the first aspect are largely compatible with the second aspect.

According to one embodiment, applicable to any one of the first and second aspects of the disclosure, the medicament container is a syringe. The medicament container typically comprises a medicament delivery member in the form of a needle.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a medicament delivery device according to embodiments of the present disclosure;

FIG. 2 is an exploded view of the medicament delivery device and a sub-assembly thereof according to embodiments of the present disclosure;

FIG. 3 is a cross-sectional view of at least a part of the sub-assembly according to embodiments of the present disclosure;

FIG. 4A is a perspective view of a braking button, a rotatable driver arrangement and a plunger rod of the sub-assembly, wherein the braking button is arranged in its first state according to embodiments of the present disclosure;

FIG. 4B is a perspective view of a braking button, a rotatable driver arrangement and a plunger rod of the sub-assembly, wherein the braking button is arranged in its second state according to embodiments of the present disclosure;

FIG. 5 is a partly exploded view of a medicament delivery device and a sub-assembly thereof according to embodiments of the present disclosure;

FIG. 6A is a perspective view of a braking button, a rotatable driver arrangement and a plunger rod of the sub-assembly in FIG. 5, wherein the braking button is arranged in its first state according to embodiments of the present disclosure;

FIG. 6B is a perspective view of a braking button, a rotatable driver arrangement and a plunger rod of the sub-assembly in FIG. 5, wherein the braking button is arranged in its second state according to embodiments of the present disclosure;

FIG. 7 is a partly exploded view of a sub-assembly according to embodiments of the present disclosure;

FIG. 8A is a cross-sectional view of a braking button and at least a part of a rotatable driver arrangement of the sub-assembly in FIG. 7, wherein the braking button is arranged in its first state according to embodiments of the present disclosure;

FIG. 8B is a cross-sectional view of a braking button and at least a part of a rotatable driver arrangement of the sub-assembly in FIG. 7, wherein the braking button is arranged in its second state according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

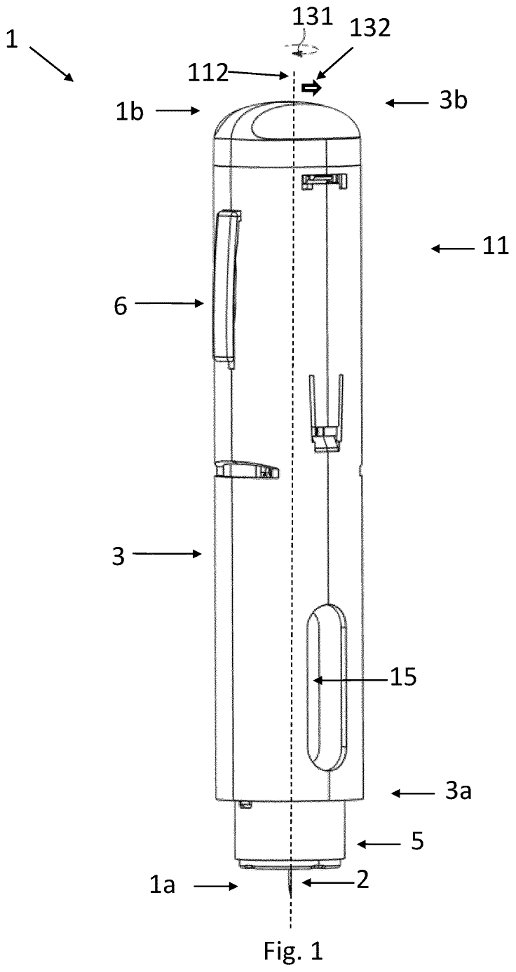

FIG. 1 shows an example of a medicament delivery device 1 such as an autoinjector of an injection device according to embodiments of the present disclosure. The medicament delivery device 1 is configured to expel medicament from a medicament container 15 via a medicament delivery member 2, here embodied as needle, to a user at a dose delivery site. The medicament delivery device 1 extends from a proximal end 1a to a distal end 1b relative to the axis 112. A needle cover 5 is arranged at the proximal end 1a and is configured to cover, and exposing, the needle 2 by moving axially relative a housing 3.

The axis 112 is in FIG. 1 a centre axis, from which a circumferential direction 131 relative to the centre axis 112 and a radial direction 132 extending radially relative to the centre axis 112, can be defined.

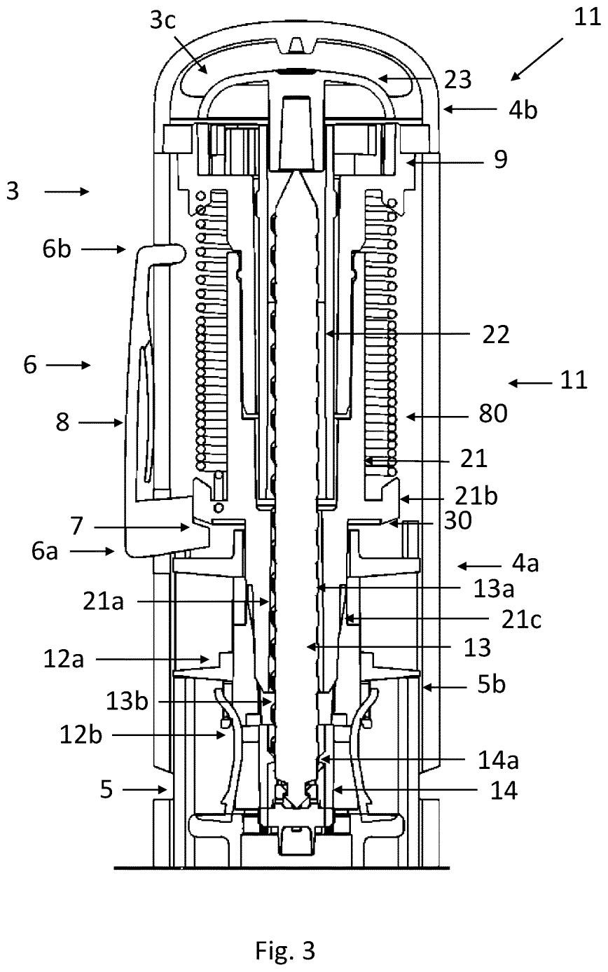

The medicament delivery device 1 comprises a sub-assembly 11, the sub-assembly comprising the housing 3 having a proximal end 3a and a distal end 3b, and a braking button 6 which will be further described with reference to FIG. 3.

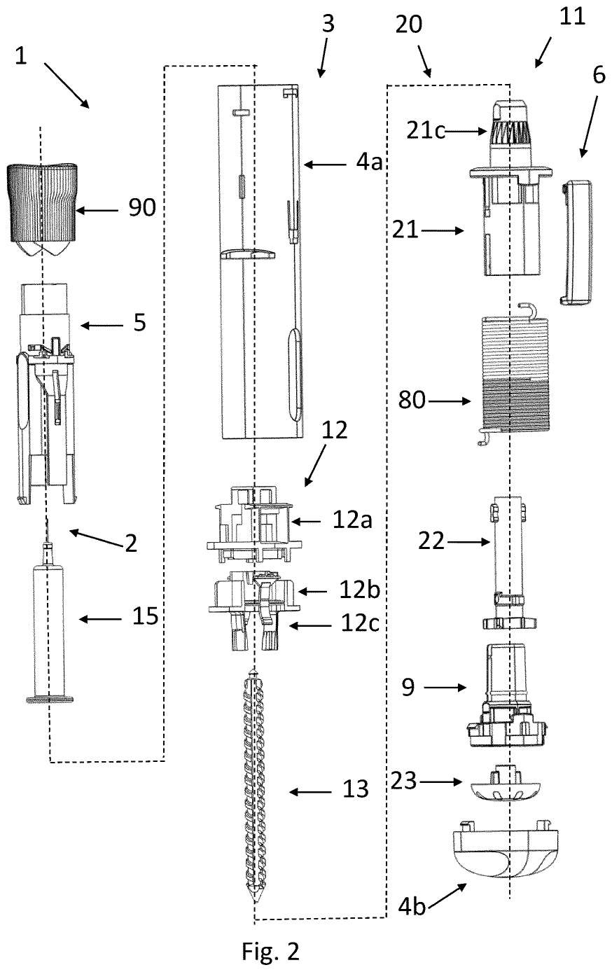

FIG. 2 is an exploded view of the medicament delivery device 1 and sub-assembly 11 of the medicament delivery device 1. The sub-assembly 11 comprises the housing 3 and the braking button 6 shown in FIG. 1. The housing 3 is here divided into a first housing part 4a and a second housing part 4b in the form of a rear cap. Moreover, the sub-assembly 11 comprises a plunger rod 13 arranged inside the housing 3, or at least inside the first housing part 4a.

The sub-assembly 11 further comprises a pre-tensioned rotatable driver arrangement 20 for the plunger rod 13. The rotatable driver arrangement 20 is operably arranged to, upon activation, rotate and thereby move the plunger rod 13 proximally to act on the medicament container 15 for expelling a medicament.

The rotatable driver arrangement 20 comprises a tubular main driver 21, a rotational lock 22 and a rotatable spinner 23. The rotatable spinner 23 is arranged in the distal portion of the sub-assembly 11 and is rotationally locked to the tubular main driver 21 via the rotational lock 22.

The rotatable driver arrangement 20 is pre-tensioned by means of a torsion spring 80. The torsion spring 80 is tensioned between the tubular main driver 21 and a distal housing part 9. The distal housing part 9 may be a separate component to the housing 3 but fixedly attached to the housing 3, e.g. to the first housing part 4a. As an alternative, the distal housing part 9 is integrated into the housing 3.

The sub-assembly 11 may further comprise an activation sleeve 12, here embodied as two separate parts, a first activation sleeve part 12a and a second activation sleeve part 12b fixedly attached to the first activation sleeve part 12a. The activation sleeve 12 may be rotationally fixed relative to the housing 3, e.g. the first housing part 4a, but may be movable in the axial direction inside the housing 3. The second activation sleeve part 12b comprises a holding arm 12c arranged to abut a locking surface 21c of the rotatable driver arrangement 20, here embodied as a locking surface 21c of the tubular main driver 21, to lock the rotatable driver arrangement 20 in its pre-tensioned state. That is, the holding arm 12c is arranged to prevent the tubular main driver 21 and the rotatable driver arrangement 20 from rotating by interacting with the locking surface 21c in a locking manner.

The sub-assembly 11 may further comprise the needle cover 5 shown in FIG. 1. The needle cover 5 is arranged to move axially relative the housing 3, and is operatively configured to distally push the activation sleeve 12 into an activation position in which the holding arm 12c is distant from the locking surface 21c of the rotatable driver arrangement 20 to thereby release the rotatable driver arrangement 20 from its pre-tensioned state. That is, as the holding arm 12c is distant from the locking surface 21c of the rotatable driver arrangement 20, the holding arm 12c no longer prevents the tubular main driver 21 and the rotatable driver arrangement 20 from rotating by the action of the torsion spring 80.

The sub-assembly 11 may further comprise an outer cap 90 being removably arranged relative to the housing 3, and/or the needle cover 5. Thus, by removing the outer cap 90, the needle 2 of the medicament container 15 may be exposed.

FIG. 3 is a cross sectional view of at least a part of the sub-assembly 11. In FIG. 3, the first housing part 4a and the second housing part 4b of the housing 3 are shown, as well as the rotatable driver arrangement 20 and its tubular main driver 21, the rotational lock 22 and the rotatable spinner 23. Moreover, in FIG. 3, the torsion spring 80 being tensioned between the distal housing part 9 and the tubular main driver 21 is shown.

In FIG. 3, the needle cover 5, such as a distal part 5b of the needle cover 5, has pushed distally on the second activation sleeve part 12b to move the activation sleeve 12 distally. Hereby, the holding arm 12c (shown in FIG. 2) is distant from the locking surface 21c, and the rotatable driver arrangement 20 is thus not locked in its pre-tensioned state. Thus, in FIG. 3, the torsion spring 80 acts on the tubular main driver 21 to rotate the tubular main driver 21. The tubular main driver 21 is rotationally fixed to the rotational lock 22, and therefore the rotational lock 22 and the rotatable spinner 23, and thereby the whole rotatable driver arrangement 20, rotates.

The tubular main driver 21 has an inner facing interacting surface 21a, and the plunger rod 13 has an outer facing interacting surface 13a. In more detail, the tubular main driver 21 is hollow and comprises a cylindrically shaped inner bore holding the plunger rod 13. The surface(s) of the inner bore facing towards the axis 112 comprises, or forms, the inner facing interacting surface 21a. Correspondingly, the plunger rod 13, typically being an elongated solid member, is configured to be moved within the cylindrically shaped inner bore of the tubular main driver 21, and has outer surface(s) facing away from the axis 112, such surface(s) comprising, or forming, the outer facing interacting surface 13a. The inner and outer facing interacting surfaces 21a, 13a are arranged to directly interact to transform a rotational movement of the tubular main driver 21 to a rotational movement of the plunger rod 13. For example, such rotational lock may comprise a male-female structure, e.g. in the form of a protrusion of the tubular main driver 21 connecting with an indentation of the plunger rod 13.

As shown in FIG. 3, the sub-assembly 11 may comprise a feeding structure 14 arranged radially outside of the plunger rod 13. Here, the feeding structure 14 is formed as a part of the second activation sleeve part 12b, and may thus be held rotationally fixed in relation to the plunger rod 13. The feeding structure 14 is arranged proximal of the rotatable driver arrangement 20, and proximal of the tubular main driver 21. The feeding structure 14 comprise an internal thread 14a configured to mate with an external thread 13b of the plunger rod 13. The external thread 13b may be arranged next to the outer facing interacting surface 13a of the plunger rod 13. For example, the outer facing interacting surface 13a of the plunger rod 13 may be formed as an elongated indentation in the external thread 13b, such elongated indentation extending axially along the plunger rod 13. Thus, as the feeding structure 14 is rotationally fixed to the housing 3, a rotational movement of the plunger rod 13 will result in that the plunger rod 13 is moved axially, or fed axially, relative to the feeding structure 14.

That is, as the torsion spring 80 acts on the tubular main driver 21 to rotate the tubular main driver 21, the plunger rod 13 is rotated and thereby moved axially in the proximal direction. In other words, the rotatable driver arrangement 20 is operably arranged to, upon activation, rotate and thereby move the plunger rod 13 proximally to act on the medicament container 15 (shown in FIG. 2) for expelling the medicament.

The speed of expelling medicament from the medicament container 15 can be reduced by activating the braking button 6 and reducing the rotation of the rotatable driver arrangement 20, as will be described in the following.

The breaking button 6 in FIG. 3 extends from a proximal end 6a to a distal end 6b, and comprises an elongated handle 8 arranged radially outside of the housing 3 from the proximal end 6a to the distal end 6b. The braking button 6 comprises an attachment surface 7 arranged radially inside the housing 3. Thus, the braking button 6 extends radially from outside of the housing 3 to inside of the housing 3. Hereby, a user may access the braking button 6 from outside of the housing 3, and apply a force to the braking button 6, whereby the braking button 6 may affect the rotational driver arrangement 20 inside of the housing 3.

As shown in FIG. 3, the attachment surface 7 of the braking button 6 is directly connected to a proximal end portion of the elongated handle 8. Hereby, the elongated handle 8 may act as a lever and improve the control of the braking button 6 to the rotational driver arrangement 20.

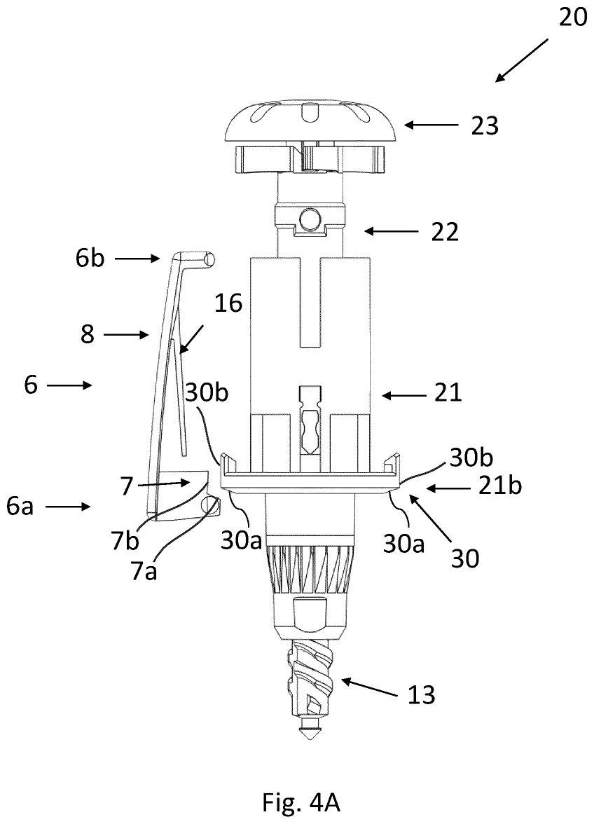

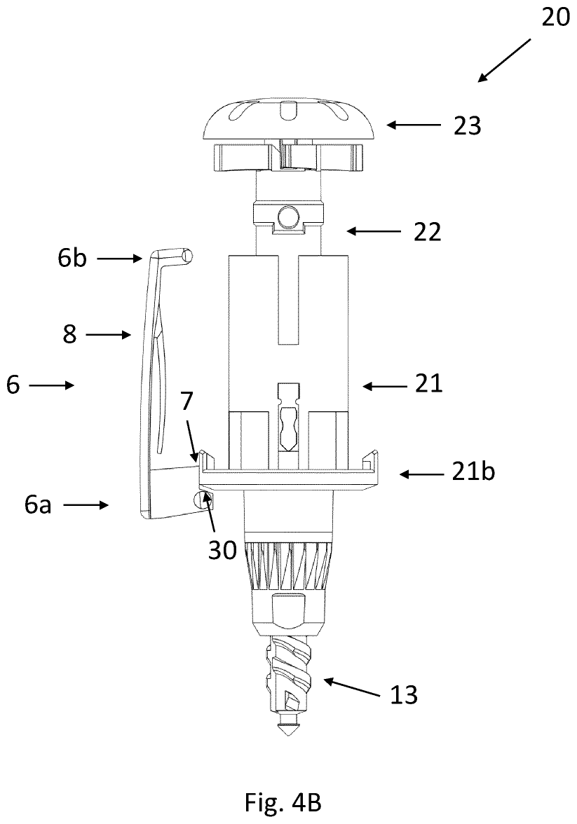

The rotatable driver arrangement 20 comprises a contacting surface 30 configured to interact with the attachments surface 7 of the braking button 6 as will now be further described in detail with reference to FIGS. 4A and 4B.

FIGS. 4A and 4B show the braking button 6, the rotatable driver arrangement 20 and the plunger rod of the sub-assembly 11 of FIG. 3. The braking button 6 is operatively arranged to, upon activation, move from a first state, shown in FIG. 4A, in which the attachment surface 7 is distant from the contacting surface 30 of the rotatable driver arrangement 20, to a second state, shown in FIG. 4B, in which the attachment surface 7 is abutting the contacting surface 30 of the rotatable driver arrangement 20. As the braking button 6 is moved from the first state to the second state, the attachment surface 7 is typically moved radially inwards. This may e.g. be achieved by pressing on the handle 8.

In the embodiment of FIGS. 4A and 4B, the contacting surface 30 of the rotatable driver arrangement 20 is arranged on the tubular main driver 21, and is comprised in an outer facing surface 21b of the tubular main driver 21. Thus, in the second state of the activation button 6 shown in FIG. 4B, the attachment surface 7 is abutting the contacting surface 30 of the tubular main driver 21 and thereby directly brake the rotational movement of the tubular main driver 21. Thus, friction between the attachments surface 7 and the contacting surface 30 results in braking of the rotational movement of the tubular main driver 21. As the tubular main driver 21 is braked, the speed of the proximal movement of the plunger rod 13 is slowed down. Hereby, the expel of medicament from the medicament container can be adapted accordingly.

As shown in FIG. 4A, the contacting surface 30 comprises a proximally facing surface portion 30a. Correspondingly, the attachment surface 7 of the braking button 6 comprises a distally facing surface portion 7a. The proximally facing surface portion 30a may be configured to abut the distally facing surface portion 7a in the second state of the braking button 6. As shown in FIG. 4A, both the proximally facing surface portion 30a and the distally facing surface portion 7a are angled (i.e. correspondingly angled), as the normal component from the respective surface portion is not extending axially. That is, the normal component of the proximally facing surface portion 30a is angled relative to the axis 112, and the normal component of the distally facing surface portion 7a is angled relative to the axis 112.

Additionally, or alternatively, the contacting surface 30 comprises an outer radial facing surface portion 30b. Correspondingly, the attachment surface 7 of the braking button 6 comprises an inner radial facing surface portion 7b. The outer radial facing surface portion 30b may be configured to abut the inner radial facing surface portion 7b in the second state of the braking button 6.

As shown in FIG. 4B, in the second state of the activation button 6, the outer radial facing surface portion 30b of the contacting surface 30 is abutting the inner radial facing surface portion 7b of the attachment surface 7 of the braking button 6. Hereby, friction between the attachments surface 7 and the contacting surface 30 results in braking of the rotational movement of the tubular main driver 21, as previously described.

However, as an alternative, or in addition to the abutment of the outer and inner radial facing surface portions 30b, 7b in the second state of the braking button 6, the proximally facing surface portion 30a of the contacting surface 30 may be abutting the distally facing surface portion 7a of the attachment surface 7 of the braking button 6 in the second state. That is, in the second state of the braking button 6, the outer and inner radial facing surface portions 30b, 7b may be abutting each other and/or the proximally and distally facing surface portions 30a, 7a may be abutting each other.

For example, by providing contact between both proximally and distally facing surface portions 30a, 7a and inner and outer radially facing surface portions 30b, 7b, friction causing the tubular main driver 21 to brake may be applied in an advantageous manner. For example, radial and/or axial forces which may cause misalignment of components inside the housing 3, e.g. the tubular main driver 21, may be reduced.

The braking button 6 may be biased to its first state by means of a spring 16, here embodied as a leaf spring. The spring 16 is typically tensioned against the housing 3, or a structure fixed to the housing 3. Hereby, the braking button 6 may be operatively arranged to, upon de-activation, move from the second state to the first state in which the attachment surface 7 is distant from the contacting surface 30 of the rotatable driver arrangement 20. For example, with reference to the handle 8, by ceasing (or reducing) the pressing of the handle 8, the spring 16 forces the braking button to move to its first state to thereby decrease, or cease, the braking of the rotational movement of the rotatable driver arrangement 20. Hereby, the speed of the proximal movement of the plunger rod 13 may be increased as compared to in the second state of the braking button 6.

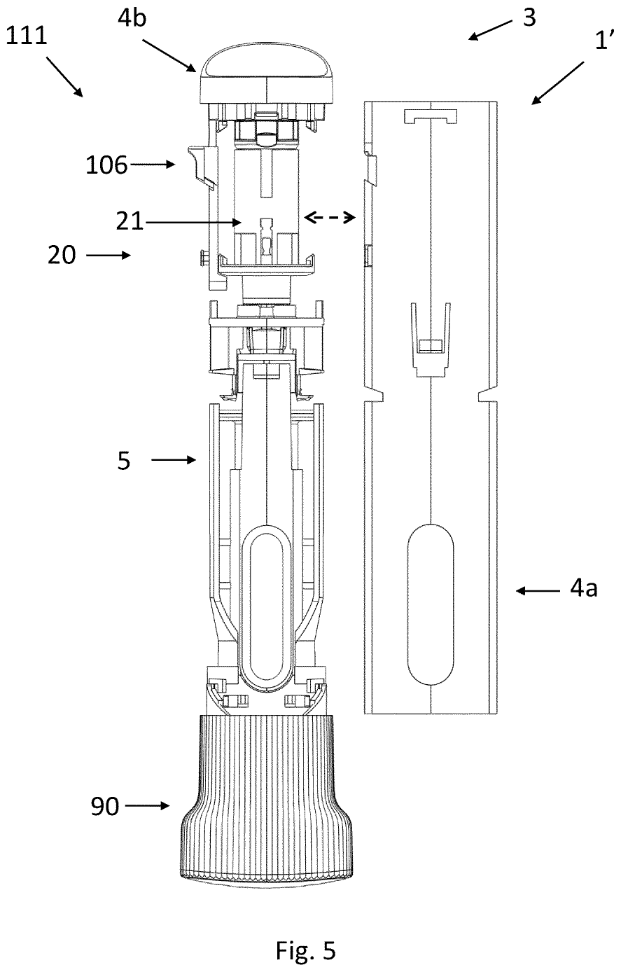

FIG. 5 is a partly exploded view of a sub-assembly 111 according to at least one embodiment. The sub-assembly 111 may be comprised in a medicament delivery device 1′.

The sub-assembly 111 of FIG. 5 is similar to the sub-assembly of FIGS. 2-3, this is why mainly the differences between the two sub-assemblies are described in the following. Thus, as can be seen in FIG. 5, the sub-assembly 111 comprises a housing 3 in the form of a first housing part 4a and a second housing part 4b, wherein the first housing part 4a has been moved laterally to disclose the internal components of the sub-assembly 111. Moreover, the sub-assembly 111 comprises a rotatable driver arrangement 120 (e.g. identical to that of the embodiments in FIGS. 2-3) comprising at least a tubular main driver 21. The sub-assembly 111 may furthermore comprise a needle cover 5 and an outer cap 90 protecting a needle of a medicament container.

The sub-assembly 111 of FIG. 5 comprises a braking button 106 in the form of a sliding button configured to move from a first state to a second state by axially sliding relative to the housing 3. The first state and the second state of the braking button 106 largely correspond to the first state and the second state of the braking button 6 of sub-assembly 11 in FIGS. 4A-4B.

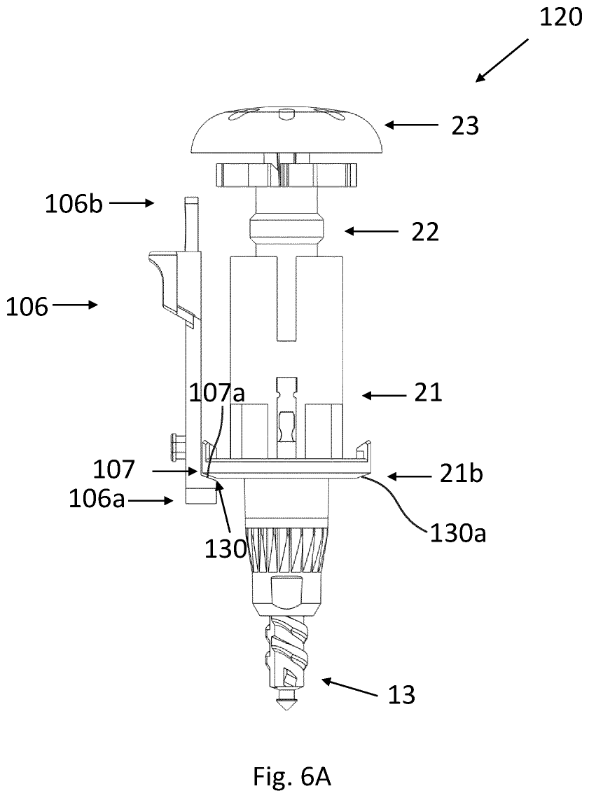

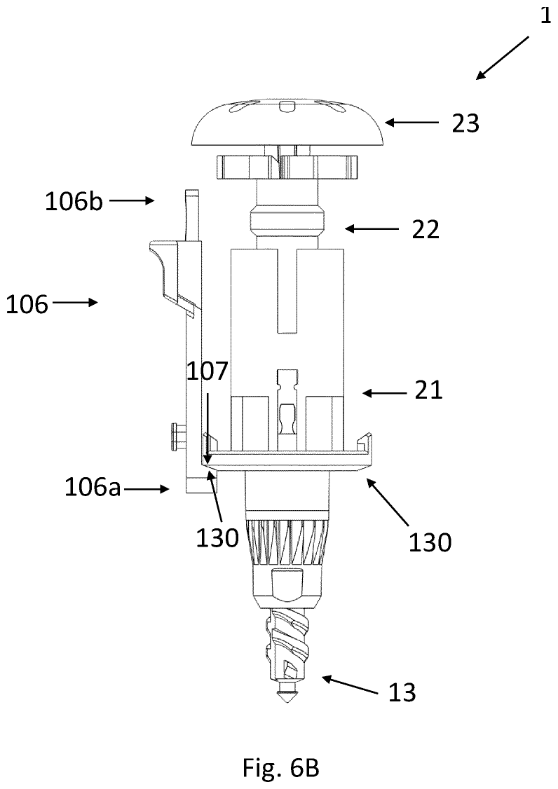

FIGS. 6A and 6B show the braking button 106, the rotatable driver arrangement 120 and the plunger rod 13 of the sub-assembly 111 in corresponding views as the sub-assembly 11 in FIGS. 4A and 4B. The braking button 106 comprises an attachment surface 107, and the tubular main driver 21 comprises a corresponding contacting surface 130. As for the braking button 6 of sub-assembly 11, the braking button 106 of sub-assembly 111 is operatively arranged to, upon activation, move from the first state, shown in FIG. 6A, in which the attachment surface 107 is distant from the contacting surface 130 of the rotatable driver arrangement 120, to the second state, shown in FIG. 6B, in which the attachment surface 107 is abutting the contacting surface 130 of the rotatable driver arrangement 120. As the braking button 106 is moved from the first state to the second state, the attachment surface 107 is typically moved axially in the distal direction. This may e.g. be achieved by sliding the braking button 106 in the distal direction.

In the embodiment of FIGS. 6A and 6B, the contacting surface 130 of the rotatable driver arrangement 120 is arranged on the tubular main driver 21, and is comprised in an outer facing surface 21b of the tubular main driver 21. Thus, in the second state of the activation button 106 shown in FIG. 6B, the attachment surface 107 is abutting the contacting surface 130 of the tubular main driver 21 and thereby directly brake the rotational movement of the tubular main driver 21. Thus, friction between the attachments surface 107 and the contacting surface 130 results in braking of the rotational movement of the tubular main driver 21. As the tubular main driver 21 is braked, the speed of the proximal movement of the plunger rod 13 is slowed down. Hereby, the expel of medicament from the medicament container can be adapted accordingly, as previously described.

As shown in FIG. 6A, the contacting surface 130 comprises a proximally facing surface portion 130a. Correspondingly, the attachment surface 107 of the braking button 106 comprises a distally facing surface portion 107a. The proximally facing surface portion 130a may be configured to abut the distally facing surface portion 107a in the second state of the braking button 106. As shown in FIG. 6A, both the proximally facing surface portion 130a and the distally facing surface portion 107a are angled (i.e. correspondingly angled), as the normal component from the respective surface portion is not extending axially. That is, the normal component of the proximally facing surface portion 130a is angled relative to the axis 112, and the normal component of the distally facing surface portion 107a is angled relative to the axis 112.

As shown in FIG. 6B, in the second state of the activation button 106, the distally facing surface portion 107a has moved distally and thus abuts the proximally facing surface portion 130a. Hereby, friction between the attachments surface 107 and the contacting surface 130 results in braking of the rotational movement of the tubular main driver 21, as previously described.

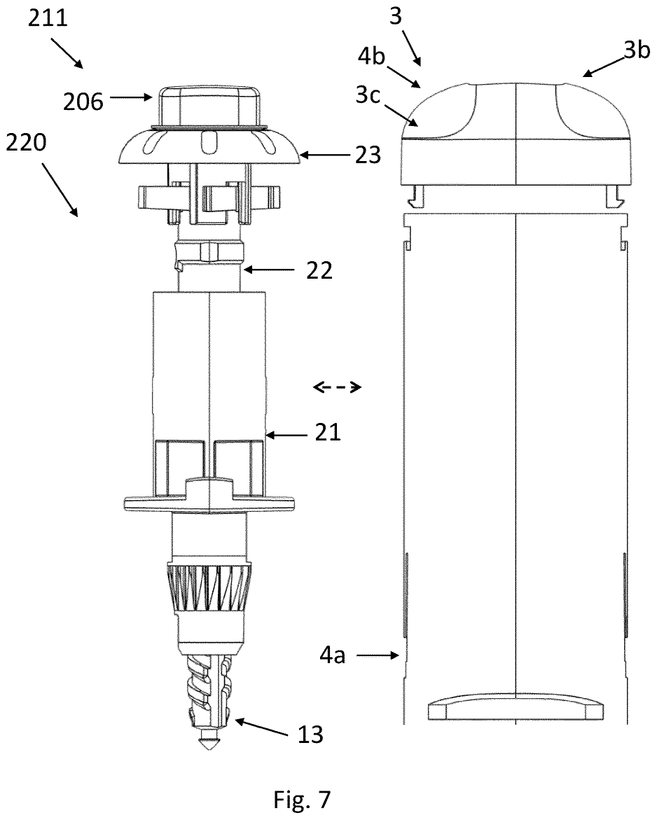

FIG. 7 is a partly exploded view of at least a part of a sub-assembly 211 according to at least one embodiment.

The sub-assembly 211 of FIG. 7 is similar to the sub-assembly 11 of FIGS. 2-3, this is why mainly the differences between the two sub-assemblies are described in the following. Thus, as can be seen in FIG. 7, the sub-assembly 211 comprises a housing 3 in the form of a first housing part 4a and a second housing part 4b, wherein the first and second housing parts 4a, 4b have been moved laterally to disclose the internal components of the sub-assembly 211. Moreover, the sub-assembly 211 comprises a rotatable driver arrangement 220 (e.g. identical to that of the embodiments in FIGS. 2-3) comprising a tubular main driver 21, a rotational lock 22 and a rotatable spinner 23. The sub-assembly 211 may furthermore comprise other components described with reference to the sub-assembly 11 of FIGS. 2-3.

The sub-assembly 211 of FIG. 7 comprises a braking button 206 arranged at the distal end 3b of the housing 3. The braking button 206 is in the form of a push-button configured to move from a protracted position in a first state to a retracted position in a second state in which the push-button is received further into the housing 3. The first state and the second state of the braking button 206 largely corresponds to the first state and the second state of the braking button 6 and braking button 106 of the previously described embodiments.

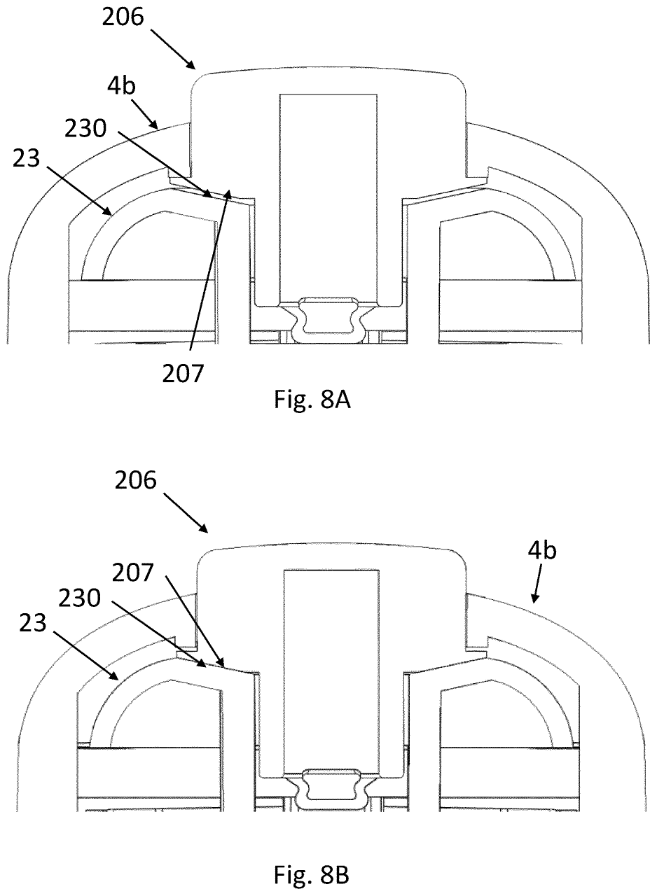

FIGS. 8A and 8B show the braking button 206, at least a part of the rotatable driver arrangement 220 and the second housing part 4b of the sub-assembly 211. The braking button 206 comprises an attachment surface 207, and the tubular spinner 23 comprises a correspondingly contacting surface 230. As for the braking button 6 of sub-assembly 11, the braking button 206 of sub-assembly 211 is operatively arranged to, upon activation, move from the first state, shown in FIG. 8A, in which the attachment surface 207 is distant from the contacting surface 230 of the rotatable driver arrangement 220, to a second state, shown in FIG. 8B, in which the attachment surface 207 is abutting the contacting surface 230 of the rotatable driver arrangement 220. As the braking button 206 is moved from the first state to the second state, the attachment surface 207 is typically moved axially in the proximal direction. This may e.g. be achieved by pushing the braking button 206 in the proximal direction.

In the embodiment of FIGS. 8A and 8B, the contacting surface 230 of the rotatable driver arrangement 220 is arranged on the rotatable spinner 23. Thus, in the second state of the activation button 206 shown in FIG. 8B, the attachment surface 207 is abutting the contacting surface 230 of the rotatable spinner 23 and thereby indirectly brakes the rotational movement of the tubular main driver 21, as the rotatable spinner 23 is rotationally locked to the tubular main driver 21 via the rotational lock 22. Thus, friction between the attachments surface 207 and the contacting surface 230 results in braking of the rotational movement of the rotatable spinner 23. As the rotatable spinner 23 is braked, the tubular main driver 21 is braked and the speed of the proximal movement of the plunger rod 13 is slowed down. Hereby, the expel of medicament from the medicament container can be adapted accordingly.

As shown in FIG. 8B, in the second state of the activation button 206, the attachment surface 207 has moved proximally and thus abuts the contacting surface 230. Hereby, friction between the attachments surface 207 and the contacting surface 230 results in braking of the rotational movement of the rotatable spinner 23.

As shown in both FIGS. 3 and 7, the housing 3 may comprise a sight window 3c arranged in a distal portion of the housing 3. Thus, the rotatable spinner 23, and its speed of rotation, may be visible in a proximal direction through the sight window 3c. Hereby, feedback of the rotatable spinner 23 may be provided to a user.

It should be noted that both braking button 106 of sub-assembly 111 and braking button 206 of sub-assembly 211 may comprise a spring correspondingly to spring 16 of braking button 6 of sub-assembly 11.

The medicament delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the medicament containers, and administrated by the medicament delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the medicament containers, and administrated by the medicament delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the medicament containers, and administrated by the medicament delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDW123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the medicament containers, and administrated by the medicament delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the medicament containers, and administrated by the medicament delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the medicament containers, and administrated by the medicament delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the medicament containers, and administrated by the medicament delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the medicament containers, and administrated by the medicament delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the medicament containers, and administrated by the medicament delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the medicament containers, and administrated by the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly for a medicament delivery device configured to expel medicament from a medicament container, the sub-assembly comprising:
a housing having a proximal end and a distal end;
a plunger rod arranged inside the housing;
a pre-tensioned rotatable driver arrangement for the plunger rod, the rotatable driver arrangement being operably arranged to, upon activation, rotate and thereby move the plunger rod proximally to act on the medicament container for expelling a medicament, the rotatable driver arrangement having a contacting surface; and
a braking button having an attachment surface, the braking button being operatively arranged to, upon activation, move from a first state in which the attachment surface is distant from the contacting surface of the rotatable driver arrangement, to a second state in which the attachment surface is abutting the contacting surface of the rotatable driver arrangement to thereby brake the rotational movement of the rotatable driver arrangement and slow down the proximal movement of the plunger rod.

17. The sub-assembly according to claim 16, wherein the braking button extends radially and/or axially from outside of the housing to inside of the housing.

18. The sub-assembly according to claim 16, wherein the rotatable driver arrangement comprises a tubular main driver holding the plunger rod, the tubular main driver having an inner facing interacting surface and the plunger rod having an outer facing interacting surface, wherein the inner and outer facing interacting surfaces are arranged to directly interact to transform a rotational movement of the tubular main driver to a rotational movement of the plunger rod.

19. The sub-assembly according to claim 18, wherein the contacting surface of the rotatable driver arrangement is arranged on the tubular main driver, and wherein in the second state of the activation button, the attachment surface is abutting the contacting surface of the tubular main driver to thereby directly brake the rotational movement of the tubular main driver.

20. The sub-assembly according to claim 19, wherein the contacting surface of the tubular main driver is comprised in an outer facing surface of the tubular main driver.

21. The sub-assembly according to claim 20, wherein the contacting surface comprises a proximally facing surface portion, and wherein the attachment surface of the braking button comprises a corresponding distally facing surface portion, such that in the second state of the activation button, the proximally facing surface portion of the contacting surface is abutting the distally facing surface portion of the attachment surface of the braking button.

22. The sub-assembly according to claim 20, wherein the contacting surface comprises an outer radial facing surface portion, and wherein the attachment surface of the braking button comprises an inner radial facing surface portion, such that in the second state of the activation button, the outer radial facing surface portion of the contacting surface is abutting the inner radial facing surface portion of the attachment surface of the braking button.

23. The sub-assembly according to claim 16, wherein the braking button comprises an elongated handle arranged radially outside of the housing, and wherein the attachment surface is directly connected to a proximal end portion of the elongated handle.

24. The sub-assembly according to claim 16, wherein the braking button is a sliding button configured to move from the first state to the second state by axially sliding relative to the housing.

25. The sub-assembly according to claim 18, wherein the rotatable driver arrangement further comprises a rotatable spinner arranged in a distal portion of the sub-assembly, the rotatable spinner being rotationally locked to the tubular main driver, wherein the contacting surface of the rotatable driver arrangement is arranged on the rotatable spinner, and wherein in the second state of the activation button, the attachment surface is abutting the contacting surface of the rotatable spinner to thereby indirectly brake the rotational movement of the tubular main driver.

26. The sub-assembly according to claim 25, wherein the braking button is a push-button configured to move from a protracted position in the first state to a retracted position in the second state in which the push-button is received further into the housing.

27. The sub-assembly according to claim 25, wherein the housing comprises a sight window arranged in a distal portion of the housing, wherein the rotatable spinner is visible in a proximal direction through the sight window.

28. The sub-assembly according to claim 16, wherein the braking button is biased to its first state such that the braking button is operatively arranged to, upon de-activation, move from the second state to the first state in which the attachment surface is distant from the contacting surface of the rotatable driver arrangement to thereby decrease, or cease, the braking of the rotational movement of the rotatable driver arrangement and increase the proximal movement of the plunger rod.

29. The sub-assembly according to claim 16, further comprising an activation sleeve rotationally fixed relative to the housing, the activation sleeve comprising a holding arm arranged to abut a locking surface of the rotatable driver arrangement to lock the rotatable driver arrangement in its pre-tensioned state.

30. A medicament delivery device for expelling medicament from a medicament container, the medicament delivery device comprising the sub-assembly according to claim 16.
